(19) United States
(12) Patent Application Publication
(10) Pub. No.: US 2024/0122694 A1
(43) Pub. Date: Apr. 18, 2024

(54) SURGICAL SYSTEM FOR AN AORTIC VALVE

(71) Applicant: Excision Medical, Inc., Malvern, PA (US)

(72) Inventors: Greg Alan WALTERS, Exton, PA (US); Chad J. SMITH, Phoenixville, PA (US)

(73) Assignee: Excision Medical, Inc., Malvern, PA (US)

(21) Appl. No.: 18/379,390

(22) Filed: Oct. 12, 2023

Related U.S. Application Data
(60) Provisional application No. 63/416,302, filed on Oct. 14, 2022.

(51) Int. Cl. A61F 2/01; A61F 2/24

(52) U.S. Cl. CPC A61F 2/013; A61F 2/0108; A61F 2/011; A61F 2/2427

(57) ABSTRACT

The present disclosure describes a surgical system for a valve in an aorta. The surgical system is configured to serve as a temporary valve and/or filter to facilitate maintenance of proper blood flow while also capturing debris of an excised leaflet as needed.

### CROSS-REFERENCE TO RELATED APPLICATIONS

The present application claims the benefit of and priority to U.S. Provisional Application No. 63/416,302, filed Oct. 14, 2022, the entire contents of which are incorporate herein by reference.

### TECHNICAL FIELD

The present disclosure relates to a surgical system for maintaining proper blood flow during or after excision of portions of an aortic valve.

### BACKGROUND

Transcatheter aortic valve replacement (TAVR) is an alternative option for the treatment of patients with severe calcific aortic stenosis. Indeed, TAVR may become the preferred therapy for all patients irrespective of surgical risk. Transcatheter heart valves (THV) may fail in the future and repeat intervention may be required. So-called redo-transcatheter aortic valve implantation (TAVI) or TAVR may lead to risks of coronary obstruction due to the leaflet of the failed valve being pushed up by the new valve and leading to obstruction of blood flow to the coronary arteries. TAVR in failed surgical bioprostheses is common. However, TAVR in failed transcatheter bioprostheses (i.e., transcatheter heart valve-in-transcatheter heart valve) will also become increasingly common. In both situations there is a risk of coronary obstruction. The risk of coronary obstruction can be predicted with the use of cardiac computed tomography. If the predicted risk of coronary occlusion is high, then percutaneous valve-in-valve intervention may be prohibitive. In some cases, the cause of the coronary obstruction is related to the leaflets of the failed surgical or transcatheter heart valve that are pushed up, preventing flow of blood to the coronary arteries.

### SUMMARY

There is a need for systems, devices, and procedures for maintaining proper blood flow during excision of portions of an aortic valve. As such, an embodiment of the present disclosure is a surgical system. The surgical system includes an elongated configurable catheter having a proximal end, and a distal end spaced from the proximal end along a distal direction. The surgical system further includes a main shaft having a terminal end. The surgical system further includes a first strut slidable in a first lumen of the elongated configurable catheter, the first strut having an intermediate section and a terminal end. The surgical system further includes a second strut slidable in a second lumen of the elongated configurable catheter, the second strut having an intermediate section and a terminal end. The surgical system further includes wherein intermediate sections of the first strut and the second strut are configured to exit the respective first and second lumens into a configuration that is spaced outwardly away from the main shaft. The surgical system further includes a blood impermeable barrier coupled at one end to the main shaft and being moveably coupled to the intermediate section of the first strut and the intermediate section of the second strut. The barrier is configured to transition between a retracted configuration and an expanded configuration.

A further embodiment of the present disclosure is a surgical system. The surgical system includes an elongated configurable catheter having a proximal end, a distal end spaced from the proximal end along a distal direction, and a configurable section spaced from the distal end. The configurable section is configured to selectively conform to a curvature of an aortic arch. The surgical system further includes a main shaft having a terminal end. The surgical system further includes a first strut slidable in a first lumen of the elongated configurable catheter, the first strut having an intermediate section and a terminal end. The surgical system further includes a second strut slidable in a second lumen of the elongated configurable catheter the second strut having an intermediate section and a terminal end. Intermediate sections of the first strut and the second strut are configured to exit the respective first and second lumens into a configuration that is spaced outwardly away from the main shaft. The surgical system further includes a blood impermeable barrier coupled at one end to the main shaft and being moveably coupled to the intermediate section of the first strut and the intermediate section of the second strut, the barrier configured to transition between a retracted configuration and an expanded configuration.

### BRIEF DESCRIPTION OF THE DRAWINGS

The foregoing summary, as well as the following detailed description of illustrative embodiments of the present application, will be better understood when read in conjunction with the appended drawings. For purposes of illustrating the present application, the drawings show exemplary embodiments of the present disclosure. It should be understood, however, that the present disclosure is not limited to the precise arrangements and instrumentalities shown in the drawings. In the drawings:

FIG. 1 is an exploded side view of a surgical system for maintaining proper blood flow during or after excision of portions of an aortic valve according to an embodiment of the present disclosure;

FIG. 2 is a side perspective view of the configurable section of the surgical system illustrated in FIG. 1 in an expanded state;

FIG. 3 is a top perspective view of the configurable section of the surgical system shown in FIG. 1 in an expanded state;

FIG. 4 is a cross-sectional view of a barrier according to an embodiment of the present disclosure;

FIG. 5 is a top perspective view of the configurable section of the surgical system illustrated in FIG. 1 in an expanded state with a barrier according to an embodiment of the present disclosure;

FIG. 6A is a cross sectional view of the barrier shown in FIG. 5 in an aorta having a large inner diameter;

FIG. 6B is a cross sectional view of the barrier shown in FIG. 5 in an aorta having a small inner diameter;

FIG. 7A is a cross sectional view of a barrier according to an embodiment of the present disclosure; and

FIG. 7B is a cross sectional view of a barrier according to an embodiment of the present disclosure.

### DETAILED DESCRIPTION OF ILLUSTRATIVE EMBODIMENTS

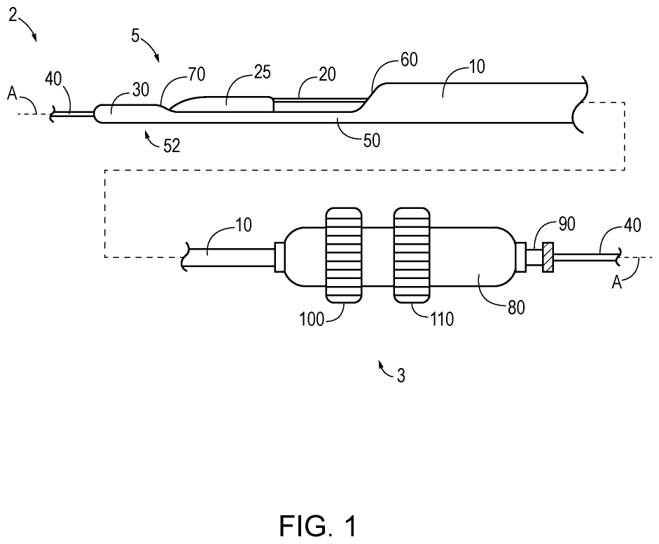

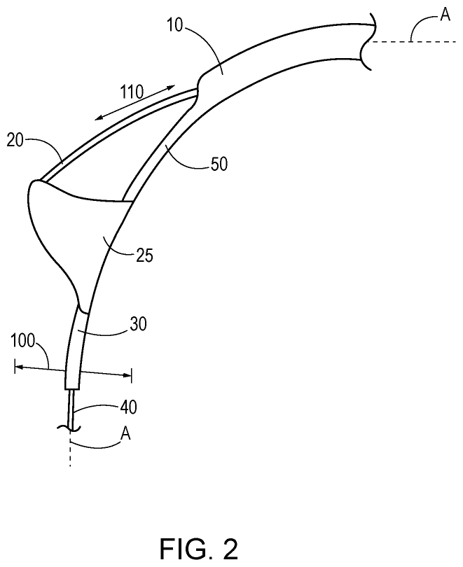

As shown in FIGS. 1-2, in the illustrated embodiments described herein, a surgical system 2 is intended to provide temporary hemodynamic protection during or after index procedures involving the cutting, excising, or manipulation of aortic valve leaflets. In cases where the surgical system is used during index procedures, the index catheter (TAVR, VIKING, etc.) is present alongside or adjacent to an expandable shield or barrier. Alternatively, the surgical system 2 may be used in cases other than aortic valve excision procedures, including, for example, in cases where aortic valve hemodynamic support is needed. The surgical system 2 may include an expandable shield or barrier that can function as a temporary one-way valve to facilitate proper blood flow. The barrier is configured to enable adequate forward flow with the left ventricle (LV) ejection of blood flow, by opening in response to blood flow. The barrier and its one-way valve type configuration are designed to limit diastolic flow in the direction of the LV.

The surgical system 2 may include a filter (not depicted) configured to capture debris as needed. The filter may include one or more movable panels (or other structures) that are responsive to fluid flow or fluid impinging the panels in order to manage blood flow in the aorta. The filter may also be used to extract and capture emboli, such as water vapor, char, smoke, oxygen, nitrogen, carbon dioxide, solids, tissue fragments, etc. In one example, the filter can be positioned to appose the aortic wall in a manner that captures particles from the forward flow ejection of the left ventricle (LV).

The surgical system may be used in combination with additional devices that are configured to a guide, capture, cut, and remove a portion of the leaflet of the valve, as described in U.S. Provisional Patent Application Ser. No. 63/324,413, filed Mar. 28, 2022, U.S. Provisional Patent Application Ser. No. 63/022,119, filed May 8, 2020, U.S. Provisional Patent Application Ser. No. 62/944,109, filed Dec. 5, 2019, U.S. Provisional Patent Application Ser. No. 63/176,507, filed Apr. 19, 2021, and U.S. patent application Ser. No. 17/782,238, filed Jun. 3, 2022, the entire contents of which are incorporated by reference to into the present disclosure.

Continuing with FIGS. 1-2, the system 2 as described herein are configured to provide access to an aorta. The surgical system 2 may therefore include one or more distinct elements designed to guide the system toward and into the aorta and position the barrier in place. More specifically, the surgical system 2 may include one or more of the following elements, either combined in a single assembly or comprising separate modular components: (a) a catheter or shaft for targeting the system 2 toward the desire tissue site; (b) a plurality of struts 20 that are responsive to fluid flow; and (c) a barrier 25 or expandable shield. The surgical system may include a handle and may include one or more actuators that are configured to control operation of and relative movement of elements ((a) through (c) above) of the system 2 in use.

The surgical system 2 is generally sized and configured for insertion into a sheath positioned in the aorta. The system 2 may include additional devices, such as guide wires, introducers, etc., to facilitate introduction of the surgical system into the aortic arch. In terms of size, the distal end and shafts of the system 2 may be sized to fit within a sheath. For example, the surgical system shaft may have an outer diameter, measured perpendicular to a central axis 1 thereof, up to about 14F. In one embodiment, the sheath may be a TAVR sheath. In another embodiment, the sheath may be an additional access sheath having a proximal end, a distal end spaced from the proximal and a lumen that extends from the proximal end to the distal end. The inner diameter of the sheath is sized to fit around a guidewire that may be at least 0.035 inches. Furthermore, the effective length of the surgical system 2, such as the portion that extends from the entry site of a patient to the target location in the aorta may vary. In some examples, the effective length may range between about 40 cm up to about 100 cm, and any intervals therebetween. In other examples, the effective length may be larger than 100 cm. Accordingly, the surgical system size and configuration could vary as needed.

The surgical system 2, and specifically the one or more elements described above ((a) through (c) and further described below) include an elongate conformable shaft or catheter 10 that engages or is coupled to the handle and is designed to extend into the aortic arch, either alone, or coupled to a guidewire, which is typically placed in the ascending arch of the aorta to provide access to an implanted valve in the aorta. The surgical system is also designed to pass through a procedural sheath. The elongate shaft may be in the form of a catheter, which includes an internal channel through which other devices and elements or may pass. Its form as a catheter is useful, as needed, when coupled with other surgical devices for access to and engagement with an implanted valve in the aorta.

As illustrated in FIGS. 1-2, the elongated catheter 10 includes a proximal end 3 and a distal end 5 that are spaced apart from each other along a central axis A. The elongated catheter 10 is therefore elongated along the central axis A. Generally, a direction from the proximal end 3 toward the distal end 5 is referred to herein as the distal direction. A direction from the distal end 5 toward the proximal end 3 is referred to herein as the proximal direction (opposite the distal direction). The elongated catheter 10 includes a distal tapered tip 30 on the distal end 5. The distal tapered tip 30 on the distal end 5 is coupled to the plurality of struts 20 at a terminal end of the plurality of struts 70 (shown in FIG. 1). The distal tapered tip 30 is further coupled to the barrier 25.

The elongated catheter 10 includes a configurable section (not numbered) spaced from the distal end 5. The configurable section is configured to selectively conform to a curvature of an aortic arch when the surgical system 2 is in an expanded configuration in the aortic arch. The configurable section is sized such that the entirety of the configurable section may be positioned in various anatomical positions including in or downstream of the left ventricular outflow track and upstream of the cerebral vasculature extending from the aortic arch.

The elongated catheter 10 includes a conformable main shaft 50, that can be selectively fixed in a curved configuration, disposed along the length of the elongated catheter 10 from the proximal end 3 to the distal end 5. The main shaft 50 includes a terminal end 52 disposed at the distal end 5. The terminal end 52 is coupled to the distal tapered tip 30 of the elongated catheter 10. The main shaft 50 extends along the central axis A parallel to the struts 20 and barrier 25. The main shaft 50 is sized and shaped to fit a guidewire lumen that extends through the main shaft 50 from the proximal end 3 to the distal end 5. The guidewire lumen is configured to receive a guidewire 40 therethrough. In the illustrated embodiment, the guidewire 40 may be at least 0.035 inches. In alternative embodiments, the guidewire may be less than 0.035 inches. The elongated catheter 10 is ideally situated between the moveable struts 20 on the outside curvature of the aorta, while the main shaft 50 is shaped to be along the inside curvature of the aorta.

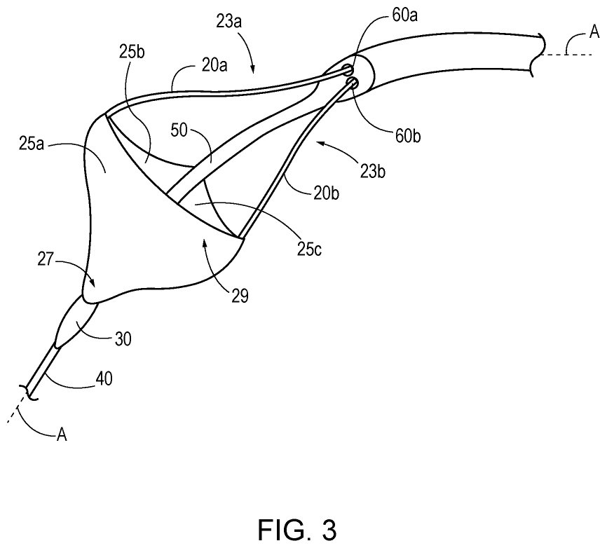

The elongated catheter 10 further includes one or more lumen 60 configured to extend longitudinally along the central axis A and parallel to the main shaft 50 and to each other. The lumen 60 partially contain the struts 20 and allow the struts to slide inside the lumen 60 from a retracted configuration to an expanded configuration. In the illustrated embodiment, the elongated catheter includes a first lumen 60A and a second lumen 60B (shown in FIG. 3). The first lumen 60A is disposed on the catheter 10 and the second lumen 60B is disposed on the catheter 10 and spaced from and parallel to the first lumen 60A.

The shafts described herein, when in the form of catheters, will generally include a shaft, an inner channel, one or more radiopaque markers, and a distal tip. One of or more catheters as described herein may have a secondary curve, a primary curve, a configurable curve, or no pre-set curves. The primary, secondary, and configurable curves are not illustrated in the drawings. The distal tip defines the distal-most end of each elongate shaft. The shaft may, for example, include an inner channel that is also sized to receive other surgical devices therethrough.

For example, the surgical system 2 can receive the guidewire 40 such that an over-the-wire technique may be used. That is, the guidewire 40 can be placed into the aorta, and the surgical system 2 may be inserted over the guidewire 40 into position via the distal tapered tip 30 and main shaft 50. In an alternative embodiment, the surgical system 2, or one or more of its shafts, may include one or more skive ports that can be used to receive the guidewire 40 therethrough. Such skive ports may be disposed toward or along an outer surface of the shaft. In yet another embodiment, the guidewire may not extend through the main shaft into the aorta. The surgical system 2, however, may still slide over or along the guidewire, but without the benefit of having the guidewire 40 cross through the main shaft.

In cross-section, a catheter may include an inner liner, a middle reinforcing layer (e.g. a braid), and an outer layer or outer jacket. In addition, the catheter may be a biaxial design that includes an additional outer layer to minimize interaction with the introducer and/or sheath and allow smoother movement of the surgical system.

The longitudinal shape of the catheter can vary as needed. For instance, the catheter can have a shape according to the Amplatz Guide that includes, but is not limited to AL-1, AL-2, AL-3, AL-4, etc. Other common shapes are possible as well. In one example, the catheter may have an outer cross-sectional dimension sized for insertion into the aorta. For instance, the catheter may be either 12 French or 14 French. However, larger or smaller sized catheters may be used in certain instances. The catheter tip, distal tip, and/or configurable section may be deflectable or bendable as needed to fix the distal portion of the catheter into position.

Continuing with FIGS. 1 and 2, the catheter 10 has at least one port 90 that extends to the inner channel. The at least one port 90 may be two or more ports as needed. The port or ports 90 are spaced a distance from the proximal end 3 that is less than a distance between the at least one port 90 and the distal end 5. In other words, they are positioned toward the proximal end 3 of the catheter 10. These ports 90 are intended to a) allow for flushing or priming the system prior to introduction to the patient and/or b) to provide for hemodynamic monitoring of the blood pressure in the ascending aorta. For instance, when the leaflets get cut, the destruction of the aortic valve may lead to decompensation of cardiac output, which is monitored by a local lumen. In one example, the system 2, may, in turn, include a luer fitting on the handle for monitoring and flushing the system.

The surgical system 2 includes a handle 80 disposed at the proximal end of the elongated catheter 10. The handle 80 may include one or more actuators disposed on the handle to control transition of the actuatable panel between and among the retracted or closed configuration and the expanded or open configuration. More specifically, in the illustrated embodiment, the surgical system 2 includes a first actuator 110 and a second actuator 100. The first actuator 110 is coupled to the struts 20. The first actuator 110 is configured to control operation of the struts 20 to permit or inhibit fluid flow through the barrier 25. In the illustrated embodiment, the actuator 110 is a knob such that when the knob is rotated, the struts 20 are configured to slide along the lumen 60 and actuate the barrier 25 to transition into an expanded configuration.

The second actuator 100 is configured to cause the configuration of the configurable section to selectively change. In the illustrated embodiment, the second actuator 100 may include one or more push-pull rods or wires coupled to either the configurable section of the elongated configurable catheter 10 or coupled to the distal tip 30. In one example, activation of one or more push-pull rods or wires causes the configurable section to curve into and out of a curved configuration. For example, one or more push-pull rods or wires may be located in a lumen inside the main shaft 50, anchored in the distal tip 30, and actuated by the handle 80. The configurable section, and a distance proximal to the configurable section may curve in at least one plane when tension is applied to the one or more push-pull rods or wires.

Referring to FIG. 3, the surgical system 2 includes a plurality of separate flexible arms or struts 20 coupled to the elongated catheter 10 and the barrier 25. In the illustrated embodiment, the surgical system 2 includes a first strut 20A and a second strut 20B. The first strut 20A is slidable along the first lumen 60A of the elongated catheter 10. The first strut 20A has a terminal end (not depicted) disposed near the distal tapered tip 30, and an intermediate section 23A that extends from the terminal end and into the first lumen 60A. The surgical system 2 further includes a second strut 20B that is slidable in the second lumen 60B. The second strut 20B has a terminal end (not depicted) disposed near the distal tapered tip 30, and an intermediate section 23B that extends from the terminal end and into the second lumen 60B. The first strut 20A and the second strut 20B are movable relative to each other and the main shaft 50. The coupling location of the first strut 20A and the second strut 20B to the main shaft 50 controls orientation of the first strut 20A and the second strut 20B. The terminal ends are coupled to the distal tapered tip 30.

The intermediate sections 23A, 23B of the struts 20A, 20B are configured to exit the respective first and second lumens 60A, 60B at a preset angle (circumferential spacing) relative to each other, at a preset shape, and at a range of distances controllable via an actuator, into a configuration that is spaced outwardly away from the main shaft 50. In one example, the preset angle of the first strut 20A, and the second strut 20B is between 90 degrees and 180 degrees. In another example, the preset angle of the first strut 20A and the second strut 20B is at least 90 degrees. This configuration enables adjusting of the struts 20A, 20B as they exit the lumens 60A, 60B to conform with various inner diameters of aortas.

Continuing with FIG. 3, the surgical system 2 further includes a barrier 25 carried by the shaft. In the illustrated embodiment, the barrier 25 has a leading end 27 defining a tapered tip and coupled to the distal tapered tip 30 and a trailing end 29 that is proximal relative to the leading end 27 and coupled, either movably or fixed, to each of the first strut 20A and the second strut 20B. The barrier 25 is movable between a retracted configuration, where the barrier 25 is collapsed toward the central axis, and an expanded configuration, where the trailing end 29 expands outwardly from the central axis A in response to a second direction of fluid flow.

The barrier 25 may be divided into one or more leaflets. In the illustrated embodiment, the barrier 25 is divided into three barrier leaflets 25A, 25B, 25C. In alternative embodiments, the number of barrier leaflets 25 may vary. The barrier 25 is coupled at one end to the main shaft 50 and the distal tapered tip 30 and is moveably coupled to the intermediate sections of the first strut and the second strut 23A, 23B respectively, such that, advancement of the first strut 20A and the second strut 20B in the distal direction causes the intermediate sections of the first strut and the second strut 23A, 23B respectively to expand outwardly in order to maintain the barrier 25 in the expanded configuration.

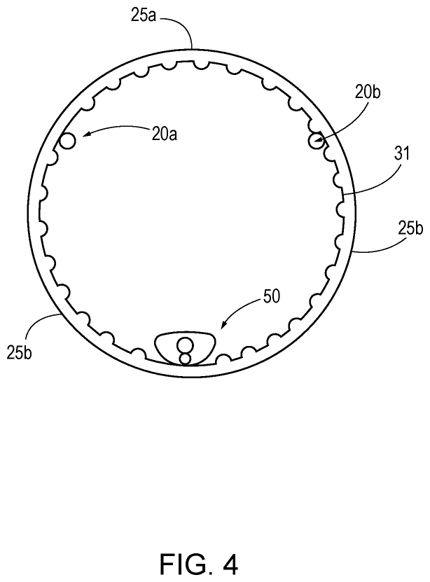

Referring to FIG. 4, in one embodiment, the barrier 25 may include longitudinal ribs 31 along an internal surface of the barrier 25. The ribs 31 may be attached or adhered to the barrier 25 in various ways that are known to one skilled in the art. In one example, the ribs 31 may be molded to the barrier 25. In another example, the ribs 31 may be adhered or machine-attached to the barrier 25. This configuration allows the barrier 25 to have increased longitudinal stiffness and may also prevent prolapsing or inverting of the barrier 25.

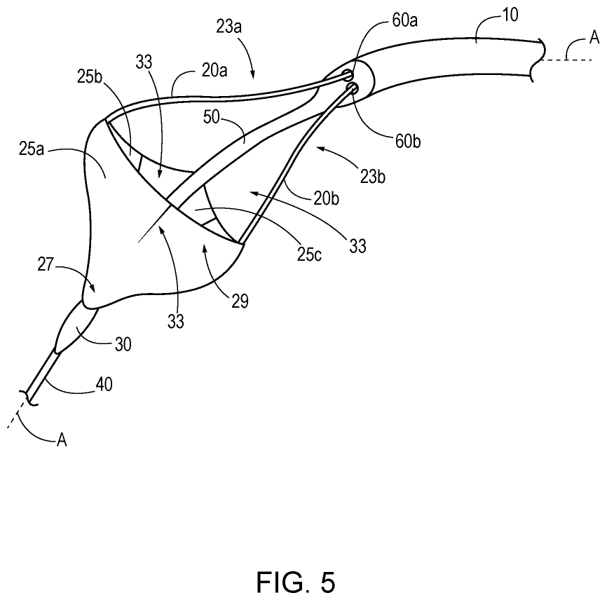

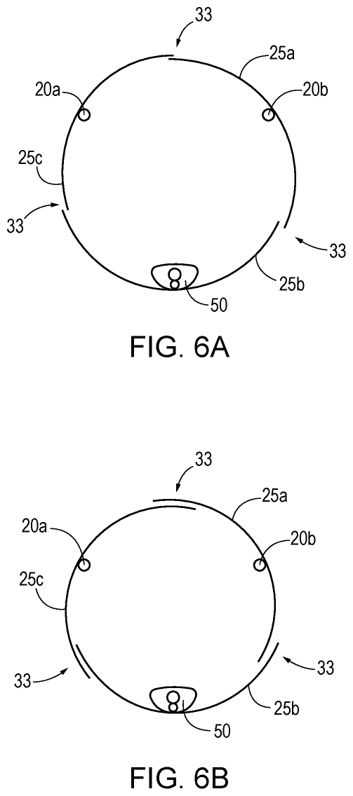

Referring to FIGS. 5-6B, in another embodiment, the barrier 25 may include one or more longitudinal slits 33 disposed at the trailing end. In the illustrated embodiment, the barrier 25 includes one slit 33 along each of the leaflets 25A, 25B, 25C. This configuration allows the slitted portion of the leaflets 25A, 25B, 25C to overlap and maintain a seal when deployed in aortas of varying internal diameters. For aortas with larger inner diameters, the leaflets 25A, 25B, 25C containing longitudinal slits 33 may have minimal overlap, as shown in FIG. 6A. For aortas with smaller inner diameters, the leaflets 25A, 25B, 25C containing longitudinal slits 33 may have larger overlap, as shown in FIG. 6B.

In another embodiment, the barrier 25 may include an opening in one or more of the leaflets 25A, 25B, 25C. The opening is configured to accommodate passage of an index catheter body (for example, TAVR, VIKING, etc.) through the barrier 25, as opposed to alongside or adjacent to the barrier 25. The opening may include a radiopaque feature to aid in angiographic guidance of the index catheter through the opening.

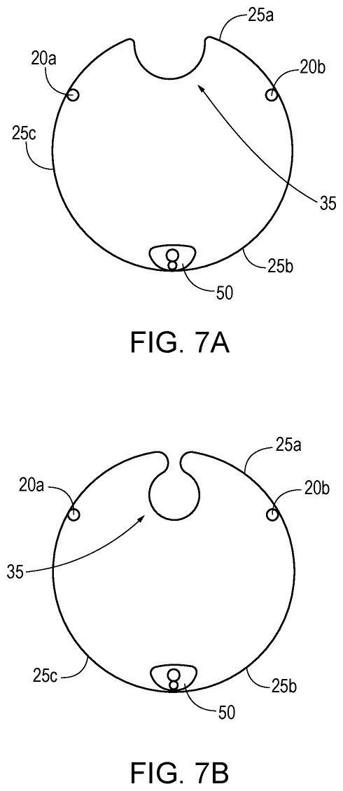

Referring to FIGS. 7A-7B, in another embodiment, the barrier 25 may include one or more channels 35 of various shapes disposed along one or more leaflets. In the illustrated embodiment, the barrier 25 includes one channel 35 along a single leaflet 25A. The one or more channels may include an opening having a radiopaque feature to aid in angiographic guidance of the index catheter through the opening. The one or more channels 35 may be a preformed shape and may include additional leaflet material spanning between two adjacent struts, or a strut and the main shaft 50, in order to accommodate an adjacent catheter body. The additional leaflet material may or may not coapt with the other leaflets 25A, 25B, 25C. This configuration allows the barrier 25 to seal around an adjacent catheter body. Furthermore, in one example, the portion of the barrier 25 between the first strut 20A and the second strut 20B may be larger than the portions of the barrier between the first strut 20A and the main shaft 50 and between the second strut 20B and the main shaft 50. In this example, the configuration allows the portion of the barrier between the first strut 20A and the second strut 20B to tuck in closer to the edges of the expandable catheter 10.

It will be appreciated by those skilled in the art that various modifications and alterations of the present disclosure can be made without departing from the broad scope of the appended claims. Some of these have been discussed above and others will be apparent to those skilled in the art. The scope of the present disclosure is limited only by the claims.

1. A surgical system, comprising:
an elongated configurable catheter;
a main shaft having a terminal end;
a first strut slidable in a first lumen of the elongated configurable catheter;
a second strut slidable in a second lumen of the elongated configurable catheter; and
a barrier coupled at the main shaft and moveably coupled to the first strut and the second strut, the barrier configured to transition between a retracted configuration and an expanded configuration.

2. The surgical system according to claim 1, further comprising an actuator coupled to the elongated configurable catheter that is configured to aid in conforming the elongated configurable catheter.

3. The surgical system according to claim 1, wherein the elongated configurable catheter further includes a main shaft and a configurable section, the configurable section being configured to selectively conform to a curvature of an aortic arch.

4. The surgical system according to claim 2, wherein the barrier is configured to transition between a retracted configuration and an expanded configuration such that the first strut and the second strut are spaced outwardly away from the main shaft.

5. The surgical system according to claim 1, wherein the first strut and the second strut each have an intermediate section and a terminal end.

6. The surgical system according to claim 5, wherein the elongated configurable catheter includes a distal tapered tip that is coupled to terminal ends of the first strut, the second strut, and the main shaft.

7. The surgical system according to claim 5, wherein the intermediate sections of the first strut and the second strut exit the respective first and second lumens at a preset angle relative to each other, a preset shape, and a preset range of distances controllable via an actuator, wherein the preset angle of the first strut and the second strut is between 90 degrees and 180 degrees.

8. The surgical system according to claim 7, wherein the preset angle of the first strut and the second strut is about 120 degrees.

9. The surgical system according to claim 5, wherein advancement of the first strut and the second strut in a distal direction causes the intermediate sections of the first strut and the second strut to expand outwardly in order to maintain the barrier in the expanded configuration,
wherein the intermediate sections of the first strut and the second strut are configured to exit the respective first and second lumens into a configuration that is spaced outwardly away from the main shaft.

10. The surgical system according to claim 1, wherein the first strut and the second strut are movable relative to each other and the main shaft.

11. The surgical system according to claim 1, wherein the main shaft includes a guidewire lumen.

12. The surgical system according to claim 3, wherein the main shaft is conformable and able to be selectively fixed in a curved configuration.

13. The surgical system according to claim 3, further comprising one or more pull rods or wires coupled to the main shaft and configured to aid in curving the main shaft.

14. The surgical system according to claim 3, wherein coupling location of the first strut and the second strut to the main shaft controls orientation of the first strut and the second strut.

15. The surgical system according to claim 1, further comprising a handle disposed on a proximal end of the elongated configurable catheter.

16. The surgical system according to claim 15, further including an actuator disposed on the handle and coupled to the first strut and the second strut, the actuator configured to cause the barrier to transition between a retracted configuration and an expanded configuration.

17. The surgical system according to claim 3, further comprising a second actuator configured to cause the configurable section to selectively change.

18. The surgical system according to claim 17, further comprising one or more push-pull rods or wires coupled to the configurable section of the elongated configurable catheter, wherein activation of the one or more push-pull rods or wires causes the configurable section to curve into and out of a curved configuration.

19. The surgical system according to claim 17, further comprising one or more push-pull rods or wires coupled to a distal tip of the elongated configurable catheter, wherein activation of the one or more push-pull rods or wires causes the configurable section to curve into and out of a curved configuration.

20. The surgical system according to claim 1, wherein the barrier is divided into one or more leaflets between the first strut, the second strut, and the main shaft.
